(19) United States
(12) Patent Application Publication
(10) Pub. No.: US 2010/0114272 A1
(43) Pub. Date: May 6, 2010

(54) MULTIPLE MICRO-WIRE ELECTRODE DEVICE AND METHODS

(76) Inventors: Sebastian Haidarliu, Rehovot (IL); Ehud Ahissar, Rehovot (IL)

Correspondence Address: Pearl Cohen Zedek Latzer, LLP, 1500 Broadway, 12th Floor, New York, NY, 10036, US

(21) Appl. No.: 12/517,197

(22) PCT Filed: Dec. 6, 2007
(86) PCT No.: PCT/IL07/01506

§ 371 Date: Dec. 20, 2009

Related U.S. Application Data
(60) Provisional application No. 60/873,269, filed on Dec. 7, 2006.
(60) Provisional application No. 60/873,950, filed on Dec. 11, 2006.

(51) Int. Cl. A61N 1/05; A61B 5/04

(52) U.S. Cl. 607/115; 600/372

(57) ABSTRACT

A device and method for targeting multiple sites of nervous tissue includes an outer elongated element (22), and inner elongated element (12), and multiple micro-wire electrodes (30) partially attached to the inner elongated element. The outer elongated element and inner elongated element are movable with respect to one another, wherein in one configuration the multiple micro-wire electrodes are positioned within the outer elongated element and in a another configuration the multiple micro-wire electrodes are positioned partially outside of the outer elongated element. Multiple micro-wire electrodes may maintain various configurations.

### FIELD OF THE INVENTION

The present invention relates to a device and methods for targeting multiple sites of nervous tissue and, more particularly, to a multi-wire electrode device and methods of use thereof.

### BACKGROUND OF THE INVENTION

In experimental and clinical neurosurgery, routine use of microelectrode recordings (MER) for monitoring neuronal electrical activity, stimulation and lesioning is on the rise. For extracellular recordings, the electrode is usually brought into the vicinity of neuronal membranes, and electrical activity of neurons up to 100 micrometers from the electrode tip can generally be recorded. Locating neurons with abnormal patterns of neuronal activity can be accomplished via MER, and can aid in curative procedures such as deep brain stimulation (DBS).

Current techniques of MER require multiple penetrations, which can cause unnecessary tissue damage and increase the duration of surgery. Moreover, only specific small targeted locations can be reached at a time, resulting in relatively small areas of recordation.

### SUMMARY OF THE INVENTION

In one embodiment, this invention provides a device for targeting multiple sites of nervous tissue comprising an inner elongated element comprising an inner elongated element distal end, an inner elongated element proximal end, and an inner elongated element body extending from said inner elongated element distal end to said inner elongated element proximal end, said inner elongated element body defining a longitudinal axis; an outer elongated element comprising an outer elongated element distal end, an outer elongated element proximal end, and an outer elongated element body extending from said outer elongated element distal end to said outer elongated element proximal end, said outer elongated element positioned concentrically with respect to said inner elongated element, wherein said inner elongated element is movable with respect to said outer elongated element along said longitudinal axis; and multiple micro-wire electrodes, each of said multiple micro-wire electrodes comprising an attached portion, said attached portion at least partially attached to said inner elongated element, and a free portion, said free portion positionable within said outer elongated element body.

In one embodiment, this invention provides a method for performing an activity in a neuronal structure, the method comprising the following steps: inserting a device comprising multiple micro-wire electrodes enclosed in an enclosure into a subject; advancing said device into close proximity with the neuronal structure; releasing said multiple micro-wire electrodes from said enclosure; spreading said multiple micro-wire electrodes into a configuration at the neuronal structure; and performing an electrode-based activity on the neuronal structure using said spread multiple micro-wire electrodes.

Unless otherwise defined, all technical and scientific terms used herein have the same meaning as commonly understood by one of ordinary skill in the art to which this invention belongs. Although methods and device similar or equivalent to those described herein can be used in the practice or testing of the present invention, suitable methods and device are described below. In case of conflict, the patent specification, including definitions, will control. In addition, the device, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1A depicts a single regular microelectrode inserted into a brain and targeting a subthalamic nucleus (STN) of the brain.

FIG. 1B depicts a multiple micro-wire electrode device inserted into the brain and targeting the STN, in accordance with embodiments of the present invention.

FIG. 2 depicts a cross-sectional illustration of a device having multiple micro-wire electrodes enclosed therein in accordance with embodiments of the present invention.

FIG. 3 depicts a cross-sectional illustration of the device of FIG. 2 with the multiple micro-wire electrodes released and spread out, in accordance with embodiments of the present invention.

FIG. 4 depicts a close-up illustration of a portion of the micro-wire electrodes of FIG. 3.

FIGS. 5A-5D depicts several stages of micro-wire electrode spreading, in accordance with embodiments of the present invention.

FIGS. 6A-F depicts lateral views of consecutive phases of a micro-wire electrode spreading.

FIGS. 7A-D depicts axial views of several stages of micro-wire electrode spreading.

FIGS. 8A-8D depicts several stages of micro-wire electrode spreading wherein micro-wire electrodes are asymmetrically arranged with respect to a longitudinal axis, in accordance with additional embodiments of the present invention.

FIG. 9 depict a flow-chart diagram illustration of a method of mapping or charting the spatial arrangement of tips of micro-wire electrodes in vitro and in vivo.

FIGS. 10A-B depicts the results of a multiple micro-wire electrode 2D and 3D mapping.

FIG. 11 represents examples of single- and multi-unit recordings from posterior medial (POm) thalamic nucleus performed by different micro-wires of a single multi-electrode. The windows represent action potentials of individual neurons (first 3 windows of each block) and of a few neurons (4^{th} window in each block) in vicinity of the electrode tip; window axes are 2 ms (x-axis), and 5 V (y-axis).

FIGS. 12A-12B illustrates effectiveness of trains with rhythmic constant (A) stimulus duration (5 Hz), and with variable (B) stimulus duration on a rat behavioral reactions (induced whisker movement); red dashed lines are stimulus applications, black curves are whisker deflections. “CONTRA” and “IPSI” are contralateral and ipsilateral, and refer to the side of the affected whiskers relative to the site of stimulation.

FIG. 13 depicts a histological illustration showing lesions in a rat brain, wherein the lesions are produced by micro-wire electrodes.

FIG. 14 depicts a histological illustration showing additional lesions in a rat brain, wherein the lesions are produced by micro-wire electrodes having a different configuration.

FIG. 15 depicts a radiovisiogram depicting eight micro-wire electrodes emerging from the tip of the outer elongated element.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE PRESENT INVENTION

In the following detailed description, numerous specific details are set forth in order to provide a thorough understanding of the present invention. It will be understood by those of ordinary skill in the art that the present invention may be practiced without these specific details. In other instances, well-known methods, procedures and components may not have been described in detail so as not to obscure the present invention.

The present invention is directed to a device and methods for targeting multiple sites of nervous tissue with a minimal number of penetrations. Specifically, the present invention can be used to generally simultaneously provide multiple micro-wire electrodes particularly arranged to target a particular volume of neuronal tissue. The principles and operation of a delivery device and methods according to the present invention may be better understood with reference to the drawings and accompanying descriptions.

It is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

This invention provides, in some embodiments, device and methods for targeting multiple sites of nervous tissue. In one embodiment, such device and methods will find application in recording neuronal activity. In another embodiment, such device and methods will find application in stimulating neuronal structure. In another embodiment, such device and methods will find application in creating lesions in neuronal structure. In another embodiment, such device and methods will find application in spatial mapping of patterns of neuronal activity. In another embodiment, such device and methods will find application in spatial mapping of abnormal neuronal activity. In another embodiment, such device and methods will find application in spatial mapping of normal neuronal activity.

In some embodiments, the device and methods of this invention are used for targeting sites of nervous tissue. In another embodiment, the nervous tissue is in the brain or brain regions. In another embodiment, the nervous tissue is in the spinal cord. In another embodiment, the nervous tissue is in peripheral nerves that branch throughout the body.

The term “neuronal activity” for the purpose of the present invention includes activity of neuronal circuits triggered by for example disease, behavior, pain, learning, fear, stress, or drugs, such activity being involved in passing an input through different neuronal structures within a circuit to process an input, where the response of the neuronal circuit is involved in the transfer of sensory information from the level of the whiskers to the level of the cortex. Each disorder or disease will have a specific circuit that will be affected. In another embodiment, the electrical activity is of a single neuron or the activity of an ensemble of neurons. Behaviors or other products of a neural system (e.g., hormones, growth factors, neurotransmitters, ions, etc.) can also be detected, and used as a feedback signal to determine the magnitude and strength of the modulating applied field. In another embodiment, neurons display variations in their membrane potential, such as action potentials, depolarization, and hyper polarization. These changes in the membrane potential can be utilized as a measure of neuronal activity.

The term “neural structure” as used herein means a structure that is part of the nervous system including, for example, the brain and spinal cord, the cranial and spinal nerves, autonomic ganglia, plexuses and the sub thalamic nucleus of a brain

In one embodiment, this invention provides a device for targeting multiple sites of nervous tissue, comprising an inner elongated element comprising an inner elongated element distal end, an inner elongated element proximal end, and an inner elongated element body extending from said inner elongated element distal end to said inner elongated element proximal end, said inner elongated element body defining a longitudinal axis; an outer elongated element comprising an outer elongated element distal end, an outer elongated element proximal end, and an outer elongated element body extending from said outer elongated element distal end to said outer elongated element proximal end, said outer elongated element positioned concentrically with respect to said inner elongated element, wherein said inner elongated element is movable with respect to said outer elongated element along said longitudinal axis; and multiple micro-wire electrodes, each of said multiple micro-wire electrodes comprising an attached portion, said attached portion at least partially attached to said inner elongated element, and a free portion, said free portion is positionable within said outer elongated element body.

In some embodiments of this invention the device comprises inner and outer elongated units. In one embodiment the inner elongated body and the inner elongated elements have a tubular shape. In another embodiment, the inner elongated body and the inner elongated body have a cylinder shape.

In another embodiment the outer elongated body and the outer elongated elements have a tubular shape. In another embodiment, the outer elongated body and the outer elongated body have a cylinder shape.

In another embodiment, the inner and the outer elongated bodies and elongated elements having the same shape. In another embodiment, the inner and the outer elongated bodies and elongated elements have a tubular shape. In another embodiment, the outer elongated element is positioned concentrically with respect to inner elongated element. In another embodiment, the inner elongated element is nestled inside of outer elongated element and is moved back and forth within outer elongated element. In one embodiment, when the inner elongated element is in its fully proximal position, proximal end is outside of outer elongated element. In another embodiment, when inner elongated element is pushed distally from proximal end, the distal end reaches distal end of outer elongated element.

In one embodiment the inner elongated element body and the outer elongated element body define the longitudinal axis. In one embodiment, the length of the inner elongated body is between 32-120 mm. In another embodiment, the length of the inner elongated body is between 30-120 mm. In another embodiment, the length of the inner elongated body is between 25-150 mm. In another embodiment, the length of the inner elongated body is between 25-150 mm. In another embodiment, the length of the inner elongated body is between 50-100 mm. In one embodiment, the length of the outer elongated body is between 22-110 mm. In one embodiment, the length of the outer elongated body is between 20-120 mm. In one embodiment, the length of the outer elongated body is between 30-160 mm. In one embodiment, the length of the outer elongated body is between 30-120 mm. In one embodiment, the length of the outer elongated body is between 25-100 mm.

In one embodiment, the outer elongated body and/or element and inner elongated body and/or element are comprised, independently from a biocompatible material, for example, but not limited to, stainless steel, titanium, titanium alloys, cobalt, chromium, or a biocompatible polymer such as polyimide.

In one embodiment, the device of this invention comprises multiple micro-wire electrodes. In another embodiment, the device comprises any number of electrodes. In another embodiment, the device comprises between 4-30 micro-wire electrodes. In another embodiment, the device comprises between 5-10 micro-wire electrodes. In another embodiment, the device comprises between 8-16 micro-wire electrodes. In another embodiment, the device comprises between 4-10 micro-wire electrodes. In another embodiment, the device comprises between 15-20 micro-wire electrodes. In another embodiment, the device comprises between 10-20 micro-wire electrodes. In another embodiment, the device comprises between 20-30 micro-wire electrodes. In another embodiment, the number of micro-wire electrodes may be any number which can reasonably fit within outer elongated element and/or body. In another embodiment, the number of micro-wire electrodes depends on the inner diameter of the outer elongated element and/or body. In another embodiment, the number of micro-wire electrodes depends on the diameter of each micro-wire electrodes.

In one embodiment, the diameter of the micro-wire electrodes is less than 40 μm.

In another embodiment, the diameter of the micro-wire electrodes is between 20-40 μm.

In another embodiment, the diameter of the micro-wire electrodes is between 25-33 μm.

In another embodiment, the diameter of the micro-wire electrodes is between 10-40 μm.

In another embodiment, the diameter of the micro-wire electrodes is between 10-20 μm.

In another embodiment, the diameter of the micro-wire electrodes is between 20-30 μm.

In another embodiment, the diameter of the micro-wire electrodes is between 30-40 μm.

In another embodiment, the diameter of the micro-wire electrodes is between 40-50 μm.

In another embodiment, the diameter of the micro-wire electrodes is between 50-60 μm.

In one embodiment, the device of this invention comprises multiple micro-wire electrodes, each of said multiple micro-wire electrodes comprising an attached portion, and a free portion, wherein said free portion is positionable within said outer elongated element body. In another embodiment, the length of the free portion is any suitable length which is necessary to define the neuronal structure and/or activity. In another embodiment, the length of the free portion is between 1-6 mm. In another embodiment, the length of the free portion is between 3-4 mm. In another embodiment, the length of the free portion is between 1-10 mm. In another embodiment, the length of the free portion is between 5-10 mm. In another embodiment, the length of the free portion is between 10-20 mm. In another embodiment, the length of the free portion of some of the multiple micro-wire electrodes differs from the length of the free portion of other micro-wire electrodes. In another embodiment, the length of the free portion depends on the use of the device of this invention. In another embodiment, the use of the device for animal studies requires different length of specific electrodes in the free portion or different total length of free portion for clinical use in humans.

In one embodiment, the multiple micro-wire electrodes are comprised of an attached portion and a free portion having tips at distal ends thereof. In another embodiment the attached portion is at least partially attached to the inner elongated element. In one embodiment attachment is accomplished by applying a small bead of superglue into distal end. In another embodiment, the attached portion of the micro-wire electrodes is attached to the inner elongated element by soldering. In another embodiment the free portion is positioned outside of inner elongated element. In another embodiment, in the closed position, the micro-wire electrodes are enclosed within outer elongated element. When inner elongated element is pushed distally, the free portion of the micro-wire electrodes exit the outer elongated element and/or body and is exposed to the environment.

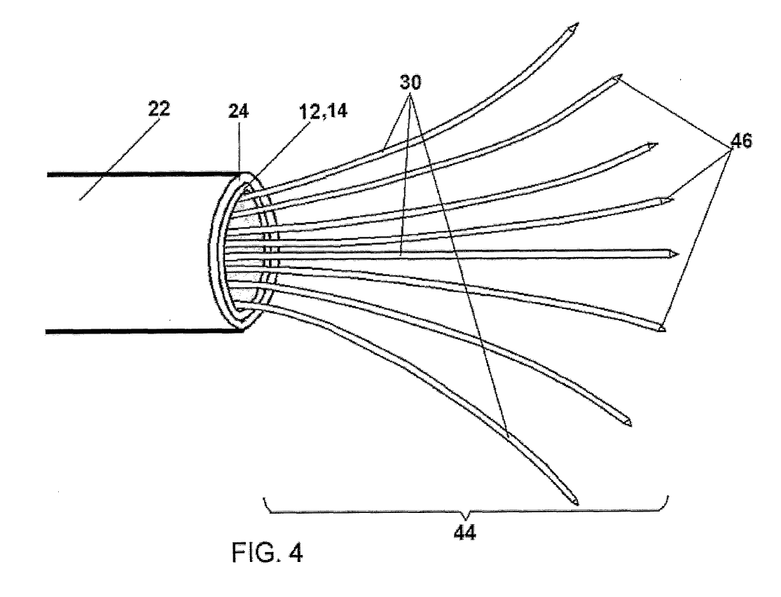

In some embodiments, the micro-wire electrodes of this invention are pre-shaped prior to their enclosure within outer elongated element and/or body. In one embodiment, pre-shaping may be accomplished by using a shape memory alloy as a material for micro-wire electrodes. In another embodiment, pre-shaping may be accomplished by using a material with high plasticity, such that deformations which are introduced to the wires prior to their enclosure within outer elongated element and/or body will remain upon release. In another embodiment, pre-shaping allows the micro-wire electrodes to be expanded or spread into a diameter (D) which is greater than a diameter of outer elongated element and/or body. In another embodiment, pre-shaping allows the micro-wire electrodes to be expanded or spread into a diameter (D) which is greater than a diameter of outer elongated element and/or body as depicted in FIG. 3. In another embodiment, the final configuration of micro-wire electrodes is tailored to the size and shape of the target area. In another embodiment, the final configuration of micro-wire electrodes may depend on the mechanical properties of the wires. In another embodiment, shaping of the free portion of micro-wire electrodes is done to accommodate a spherical, ellipsoidal, biconvex lens or any other shape of tissue. In another embodiment, FIGS. 3 and 4 depicts the shaping of the free portion 44 of micro-wire electrodes 30.

In another embodiment, pre-shaping allows the micro-wire electrodes to be expanded or spread into a diameter (D) which is greater than a diameter of outer elongated element and/or body. In another embodiment the micro-wire electrodes may be expanded or spread into a diameter (D) in the range of between 0.4 mm to 6 mm. In another embodiment the micro-wire electrodes may be expanded or spread into a diameter (D) in the range of between the inner diameters of the outer elongated element to 20 mm. In another embodiment the micro-wire electrodes may be expanded or spread into a diameter (D) in the range of between 0.4 mm to 20 mm. In another embodiment the micro-wire electrodes may be expanded or spread into a diameter (D) in the range of between 0.4 mm to 10 mm. In another embodiment the micro-wire electrodes may be expanded or spread into a diameter (D) in the range of between 1 mm to 10 mm. In another embodiment the micro-wire electrodes may be expanded or spread into a diameter (D) in the range of between 1 mm to 20 mm. In another embodiment the micro-wire electrodes may be expanded or spread into a diameter (D) in the range of between 5 mm to 10 mm. In another embodiment the micro-wire electrodes may be expanded or spread into a diameter (D) in the range of between 5 mm to 15 mm.

According to further features in embodiments of the present invention, the device comprises an outer elongated body and the device further comprises an inner elongated body positioned within and movable with respect to the outer elongated body. The inner elongated body connected to the multiple micro-wire, electrodes, and the multiple micro-wire electrodes are released by pushing the inner elongated body. The micro-wire electrodes in some embodiments are spread outside the outer elongated body. The spreading may include pre-shaping the multiple micro-wire electrodes into a pre-determined configuration prior to enclosing the multiple micro-wire electrodes in the enclosure, such that upon release of the multiple micro-wire electrodes from the enclosure, the pre-shaped multiple micro-wire electrodes maintain the pre-determined configuration.

In one embodiment, advancing the device into close proximity with a targeted neuronal structure, releasing multiple micro-wire electrodes from outer elongated element and/or pulling the multiple micro-wire electrodes back into the outer elongated element, is done manually or by automated means. In one embodiment, the free portion is releasable from the outer elongated element upon movement of the inner elongated element with respect to the outer elongated element. In another embodiment, the movement is conducted manually. In another embodiment, the movement of the inner elongated element is automated. In another embodiment, the movement of the inner elongated element is controlled by a computer. In another embodiment it is done by automated means, wherein the device is attached to a control system, where the rate and distance of the movement of the inner element and/or body are controlled. In one embodiment the control system includes an automated feedback mechanism. In one embodiment the feedback mechanism involves the use of an optical microscope. In one embodiment the feedback mechanism is based on electrical input. In another embodiment, the movement of the inner elongated element depends on the target area. In another embodiment, the free portion is releasable in such a way that the microelectrodes can reach the target area. In another embodiment, using multiple micro-wire electrodes, and enabling pre-determination of a configuration of the multiple wires, a larger volume of the target area can be accessed. In one embodiment, the outer elongated body is a guiding catheter or other guiding device. In another embodiment, the outer elongated body comprises a lumen for positioning of the micro-wire electrodes there through.

In one embodiment, the multiple micro-wire electrodes are made of an iridium-platinum alloy. In another embodiment, the multiple micro-wire electrodes are made of gold. In another embodiment, the multiple micro-wire electrodes are made of platinum. In another embodiment, the multiple micro-wire electrodes are made of optical sieves. In another embodiment, any effective electrodes can be used for recording, sensing, and as field electrodes. In one embodiment electrode material can be, but is not limited to, metal, steel, activated iridium, platinum, iridium-platinum, iridium oxide, titanium oxide, silver chloride or gold chloride. In another embodiment, the electrodes are insulated by glass or lacquer, or by materials used in silicon microelectronics, including tetrode or other multi-electrode arrays or bundles, multi-channel or ribbon devices. In another embodiment, the micro-wire electrodes are each individually insulated, with, for example, polyimide coating of each of multiple-wire electrodes. In one embodiment, the micro-wire electrodes are comprised of a biocompatible material which is corrosion resistant and stiff with transverse elasticity. In another embodiment, suitable wires are available, for example, from California Fine Wire, Grover Beach, Calif., catalog items Inconel 625 S-ML and Inconel 718 S-ML. In another embodiment, the electrodes are metal substrate, such as noble metal (e.g., Au, or Pt), ferrous steel alloy, stainless steel, tungsten, titanium, Si microprobe or other suitable substrate, that are coated with a film of an insulator such as iridium oxide in one embodiment, to produce an effective electrode. Any suitable method to prepare the coating can be used, including, but not limited to, an activation process, to form activated iridium oxide films, thermal decomposition to form thermal iridium oxide films, reactive sputtering to form sputtered iridium oxide films, evaporation of a coating material onto the electrodes or electrodepositing to form electrodeposited iridium oxide or other insulating films. Electroless deposition, dipping into a coating material or self-assembly of organic monolayer can be used to coat the electrode in some embodiments. Electrodes can be coated by immersing the electrodes into a polymer mixture wherein the polymers self-assemble on the electrode. In another embodiment the electrodes are immersed in a solution of monomers. A chemical polymerization reaction is then initiated followed by precipitation or adhesion of the formed polymers onto the electrode. In another embodiment, the activity is measured from one or more multiple micro-wire electrodes, preferably two or more. In another embodiment, the activity is recorded from several regions of the neural system in order to characterize its activity. In another embodiment, recordings of intracellular, extra-cellular, or a combination thereof, can be analyzed separately, or together. In another embodiment, the electrodes can be AC- or DC-coupled.

In some embodiments, the multiple micro-wire electrodes are made of a shape memory alloy for pre-shaping of the free portion. In other embodiments, the multiple micro-wire electrodes are made of a material having high plasticity such that pre-deformation of the material results in permanent deformation.

In one embodiment the micro-wire electrodes of this invention comprise a tip at the distal end of the micro-electrode wires. In one embodiment the tip has a sharp structure. In one embodiment the tip of the micro-wire electrode is conical in shape. In one embodiment the wire electrode tips have a disc type structure. In one embodiment such disc shaped tip prevents tissue scratching. In one embodiment disc shaped tip helps positioning of the microelectrode. In one embodiment a disc shaped tip helps to control current density and electrical activity. In one embodiment the tip has a round shape structure. In another embodiment, the electrodes can have relatively large tips with low resistance to detect activity from a number of neuronal elements within the neural system.

In another embodiment, smaller tipped electrodes can be used for monitoring activity from single neurons or smaller populations.

In one embodiment the tip of the micro-wire electrode is polished. In one embodiment the tip of the micro-wire electrode is cleaned. In one embodiment the tip of the micro-wire electrode is cleaned chemically. In one embodiment the tip of the micro-wire electrode is cleaned by immersing the electrode in an oxidizing solution. In one embodiment the oxidizing solution comprises an acid. In one embodiment the oxidizing solution comprises hydrochloric acid. In one embodiment the oxidizing solution comprises hydrogen peroxide. In one embodiment the oxidizing solution comprises hydrogen peroxide and ammonia. In one embodiment the micro-wire electrodes are rinsed with water after cleaning. In one embodiment the micro-wire electrodes are sterilized prior to measurement.

In one embodiment, the weight of the device does not exceed 2 gr. In one embodiment, the weight of the device is in the range of between 1-2 gr. In one embodiment, the weight of the device is in the range of between 1-3 gr. In one embodiment, the weight of the device is in the range of between 0.5-2 gr.

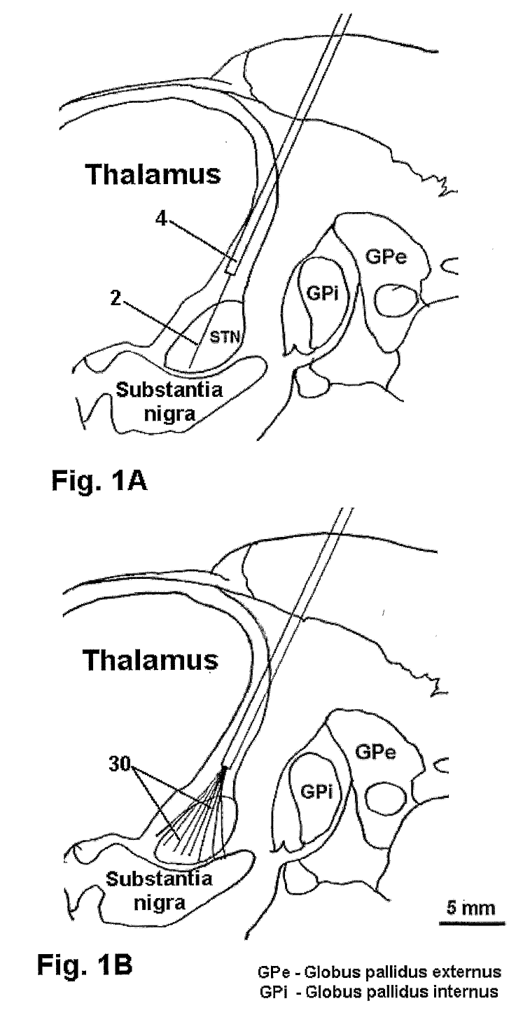

One embodiment of the device of this invention is depicted in FIG. 1B. FIG. 1B depicts a multiple micro-wire electrode inserted into the brain and targeting the sub-thalamic nucleus (STN), in accordance with embodiments of the present invention. As shown in FIG. 1B, each of the multiple micro-wires 30 may be angled differently, resulting in a spread of electrodes in the target area. By using multiple micro-wire electrodes, and by enabling pre-determination of a configuration of the multiple wires, a larger volume of the target area can be accessed.

In contrast, FIG. 1A depicts a single regular microelectrode inserted into a brain and targeting a sub-thalamic nucleus (STN) of the brain. As shown in FIG. 1A, a regular microelectrode 2 is housed in an enclosure 4. Enclosure 4 may be a guiding catheter or other guiding device, and generally includes a lumen for positioning of the microelectrode 2 there through. Since microelectrode 2 is a single electrode, only one small portion of the target area (in this case the STN) is reached. In order to provide greater coverage of the target area, enclosure 4 would need to be repositioned such that microelectrode 2 is able to subsequently reach a different area. This would need to be repeated as many times as necessary to provide sufficient coverage of the target area. Each such repetition can potentially cause damage in the brain, can result in prolong surgery, and may lead to stroke and/or hemorrhaging.

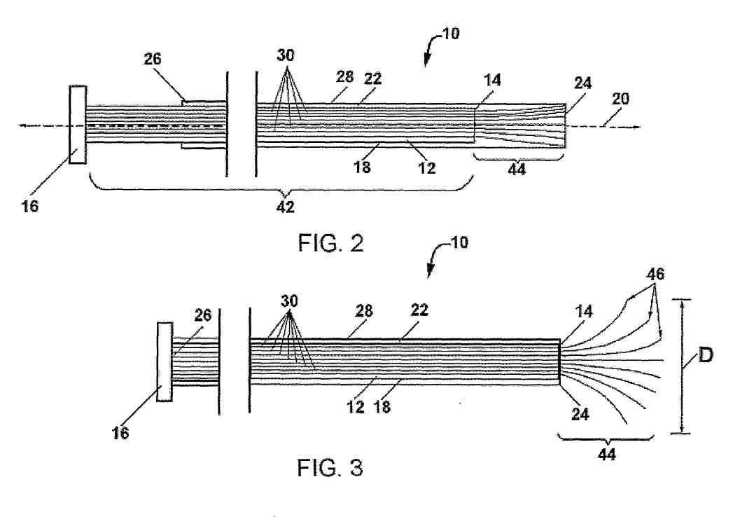

One embodiment of the device of this invention is depicted in FIGS. 2 and 3. FIG. 2 depicts a device 10 having multiple micro-wire electrodes enclosed therein. Device 10 includes an inner elongated element 12 having a distal end 14, a proximal end 16 and a body 18 extending from distal end 14 to proximal end 16. Body 18 defines a longitudinal axis 20. Device 10 further includes an outer elongated element 22 having a distal end 24, a proximal end 26 and a body 28 extending from distal end 24 to proximal end 26. Both inner elongated element 12 and outer elongated element 22 are of any suitable shape, and in some embodiments are tubular. Outer elongated element 22 is positioned concentrically with respect to inner elongated element 12, and is movable with respect thereto along longitudinal axis 20. Thus, inner elongated element 12 is nestled inside of outer elongated element 22 and may be moved back and forth within outer elongated element 22. When inner elongated element 12 is in its fully proximal position, proximal end 16 is outside of outer elongated element 22, as shown in FIG. 2. When inner elongated element 12 is pushed distally from proximal end 16, distal end 14 reaches distal end 24 of outer elongated element 22, as shown in FIG. 3.

One embodiment of the device of this invention is depicted in FIGS. 2 and 4. Device 10 further includes multiple micro-wire electrodes 30 which are positioned through inner elongated element 12. Multiple micro-wire electrodes 30 are comprised of an attached portion 42 and a free portion 44 having tips 46 at distal ends thereof. Attached portion 42 is at least partially attached to inner elongated element 12, for example, by applying a small bead of superglue into distal end 14. Free portion 44 is positioned outside of inner elongated element 12, and in a closed position is enclosed within outer elongated element 22, as shown in FIG. 2. When inner elongated element 12 is pushed distally, free portion 44 of micro-wire electrodes 30 exits outer elongated element 22 and is exposed to the environment. A closer view of free portion 44 of micro-wire electrodes 30 is depicted in FIG. 4. Free portion 44 may be any suitable length, and in some embodiments is within a range of 1-6 mm and more specifically in a range of 3-4 mm. In other embodiments, free portion 44 of at least some of multiple micro-wire electrodes 30 differs in length from free portion 44 of others of the multiple micro-wire electrodes 30.

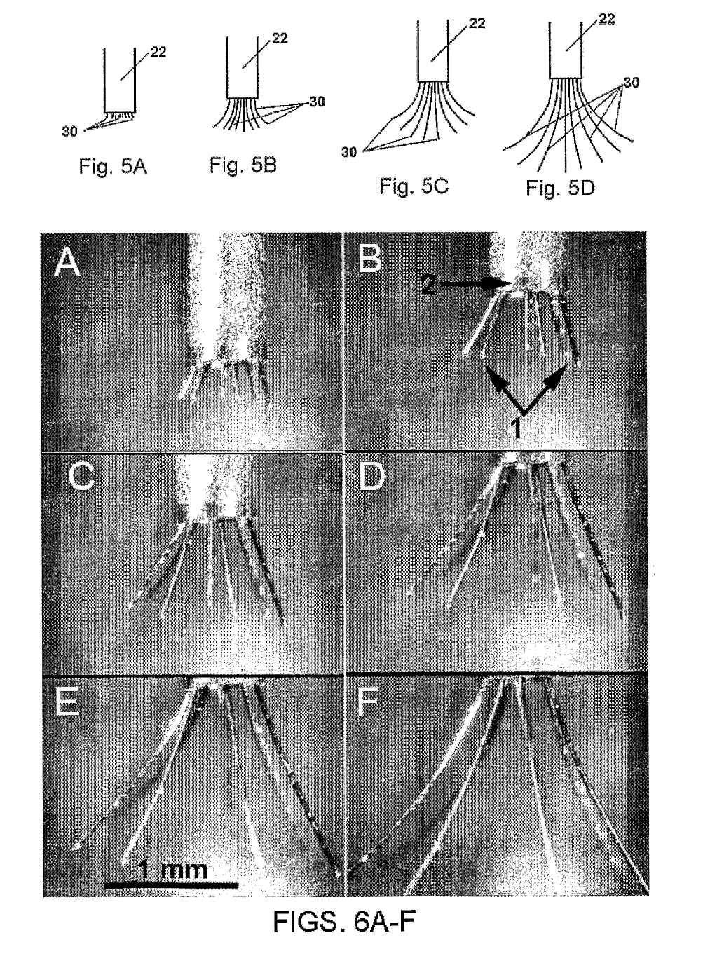

One embodiment of the device of this invention is depicted in FIGS. 5-7. In one embodiment, FIGS. 5A-5D, present schematic illustrations of several stages of micro-wire electrode 30 spreading. As shown in FIG. 5A, micro-wire electrodes 30 are initially packed into outer elongated element 22. As micro-wire electrodes 30 are released, they gradually begin to maintain a spread configuration, as seen in FIG. 5B and FIG. 5C. Finally, the fully extended and spread configuration is accomplished, as shown in FIG. 5D. Micro-wire electrodes 30 may also be retracted into outer elongated element 22, reversing the steps shown in FIGS. 5A-5D. In one embodiment FIGS. 6A-F and 7A-D present, lateral and axial views, respectively, of several stages of micro-wire electrode spreading in experimental conditions.

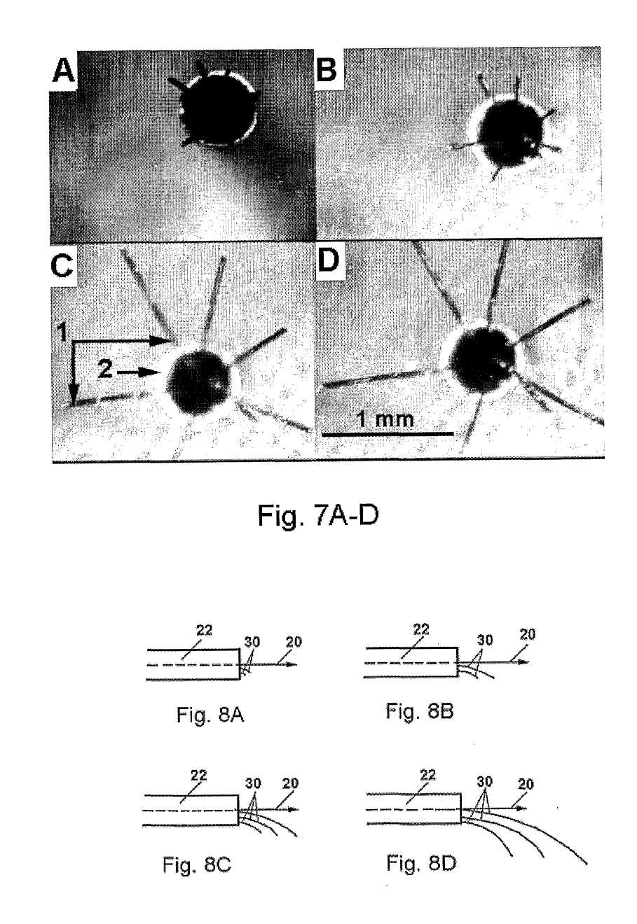

One embodiment of the device of this invention is depicted in FIG. 8. In another embodiment FIGS. 8A-8D, present illustrations of several stages of micro-wire electrode 30 spreading wherein micro-wire electrodes 30 are asymmetrically arranged with respect to longitudinal axis 20. Initially, multiple micro-wire electrodes 30 are contained within outer elongated element 22, as shown in FIG. 8A. As micro-wire electrodes begin to be pushed forward (distally), the longer wires begin to appear, while shorter wires remain within outer elongated element 22, as shown in FIG. 8B. Shorter wires begin to appear upon further advancement of micro-wire electrodes 30 (or more specifically, upon further advancement of inner elongated element 12 with respect to outer elongated element 22), as shown in FIG. 8C. When micro-wire electrodes 30 have been fully released, they may be of differing lengths, as shown in FIG. 8D. After finishing one set of recordings and/or stimulations, micro-wire electrodes 30 may be retracted into outer elongated element 22, which can then be turned about longitudinal axis 20. Micro-wire electrodes 22 can then be re-introduced to achieve a second set of recordings/stimulations in a different direction from the first set, without having to remove and reinsert the entire device 10.

In one embodiment, this invention provides a method for performing an activity in a neuronal structure, the method comprising: inserting a device comprising multiple micro-wire electrodes enclosed in an enclosure into a subject; advancing said device into close proximity with the neuronal structure; releasing said multiple micro-wire electrodes from said enclosure; spreading said multiple micro-wire electrodes into a configuration at the neuronal structure; and performing an electrode-based activity on the neuronal structure using said spread multiple micro-wire electrodes.

In one embodiment, this invention provides a method comprising the use of a device comprising an enclosure. In another embodiment the enclosure is an outer tube and the device further comprises an inner tube positioned within and movable with respect to said outer tube, said inner tube connected to said multiple micro-wire electrodes, and the multiple wire electrode are released by pushing said inner tube.

In one embodiment, this invention provides a method comprising the use of a device wherein the method comprises the step of spreading the multiple micro-wire electrodes of the device into a configuration at the neuronal structure. In another embodiment, the spreading comprises pre-shaping said multiple micro-wire electrodes into a pre-determined configuration prior to enclosing said multiple micro-wire electrodes in said enclosure, such that upon said releasing of said multiple micro-wire electrodes from said enclosure, said pre-shaped multiple micro-wire electrodes maintain said pre-determined configuration.

In another embodiment, the method for performing an activity in a neuronal structure comprises the use of the device of this invention. In another embodiment, the device of this invention comprises an inner elongated element comprising an inner elongated element distal end, an inner elongated element proximal end, and an inner elongated element body extending from said inner elongated element distal end to said inner elongated element proximal end, said inner elongated element body defining a longitudinal axis; an outer elongated element comprising an outer elongated element distal end, an outer elongated element proximal end, and an outer elongated element body extending from said outer elongated element distal end to said outer elongated element proximal end, said outer elongated element positioned concentrically with respect to said inner elongated element, wherein said inner elongated element is movable with respect to said outer elongated element along said longitudinal axis; and multiple micro-wire electrodes, each of said multiple micro-wire electrodes comprising an attached portion, said attached portion at least partially attached to said inner elongated element, and a free portion, said free portion positionable within said outer elongated element body.

In one embodiment, this invention provides a method for performing an activity in a neuronal system. In one embodiment, this invention provides a method for performing an activity in a neuronal structure. In another embodiment, the activity comprises recording the neuronal activity. In another embodiment, the activity comprises stimulating the neuronal structure. In another embodiment, the activity comprises rhythmic or variable stimulation of deep brain structures with simultaneous registration of behavioral responses. In another embodiment, the activity comprises creating lesions in the neuronal structure. In another embodiment, the activity comprises spatial mapping of patterns of neuronal activity. In another embodiment, the activity comprises spatial mapping of patterns of abnormal neuronal activity. In another embodiment, the activity comprises spatial mapping of patterns of normal neuronal activity.

In one embodiment, the methods of this invention comprise advancing the device of this invention into close proximity with the neuronal structure. In another embodiment, the distance between the tip of the device and the neuronal structure is between 20-150 μm. In another embodiment, the distance is between 50-100 μm. In another embodiment, the distance is between 20-50 μm. In another embodiment, the distance is between 50-70 μm. In another embodiment, the distance is between 70-250 μm. In another embodiment, the distance is between 100-200 μm.

In one embodiment, spatial mapping is performed prior to inserting the device into the subject, by releasing the multiple micro-wire electrodes from the enclosure, spreading the multiple micro-wire electrodes into a configuration, and determining a three-dimensional map of tips of the multiple micro-wire electrodes wherein after inserting the device into the subject, the spreading results in a similar configuration as the spreading prior to the insertion, thereby enabling spatial mapping of neuronal activity onto the determined three-dimensional map.

In one embodiment, the methods and/or device of this invention comprise mapping and/or charting the spatial arrangement of the multiple micro-wire electrodes of this invention prior to inserting the device into a subject. In another embodiment, prior to inserting the device into a subject, the three-dimensional arrangement of the tips of the micro-wire electrodes is determined by inserting the device into a solution or a gel. In another embodiment, the solution is an aqueous solution. In another embodiment the solution is a 0.95% NaCl aqueous solution. The multiple micro-wire electrodes are spread apart manually or with a micro-drive into a desired configuration and a current is introduced into each of the micro-wire electrodes consecutively until all of micro-wire electrodes have been stimulated. Gas bubbles are formed at the tips of the micro-wire electrodes. These gas bubbles are detected for example, but not limited to, by using an optoelectronic setup with two digital microscopes (for example, Labomed Digi Star, Mel Sobel Microscopes Ltd. International Falls, Minn.) rebuilt to obtain a horizontal orientation of the objective optical axes, and positioned so that their optical axes form a 90° angle in horizontal plane. When all of the bubbling micro-wire electrodes have been detected and mapped, two 2-D images for each electrode with gas bubbles are prepared. The micro-wire electrodes are retracted into outer elongated element and/or body, the device is removed from solution, and a 3-D image is computed.

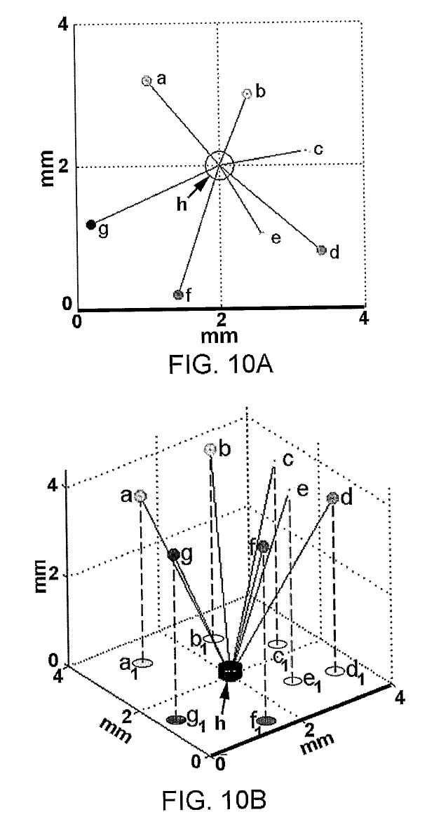

In one embodiment, creation of the 3-D image is done using two digital microscopes positioned orthogonal to one another. Each of the digital microscopes takes an image of the gas bubbles. These images are sent to a processor, which creates a 3-D image. In one embodiment, the processor is a PC or other type of computer, having 3-D creation software such as 3D-Doctor. In another embodiment, FIGS. 10A and 10B demonstrate examples of 2D and 3D, respectively, mapping of the tips of a micro-wire electrode, produced by using a MATLAB program.

In one embodiment, the methods and/or device of this invention comprise inserting multiple micro-wire electrodes into a subject. In another embodiment, the subject is human. In another embodiment, the subject is a mammal. In another embodiment the device is inserted into a subject by a surgery procedure. In another embodiment, any animal source of material is suitable, including neural systems of invertebrates, such as mollusks, arthropods, insects, etc., vertebrates, such as mammals, humans, non-human mammals, great apes, monkeys, chimpanzees, dogs, cats, rats, mice, etc. In another embodiment, the neural system comprises the sub-thalamic nucleus of a brain. In another embodiment, the neural system comprises the spinal cord. In another embodiment, the neural system comprises the nerves that branch throughout the body. In another embodiment, the neural system comprises the cranial and spinal nerves, autonomic ganglia and plexuses. In another embodiment preferred target regions include, but are not limited to, neocortex, sensory cortex, motor cortex, frontal lobe, parietal lobe, occipital lobe, temporal lobe, thalamus, hypothalamus, limbic system, amygdala, septum, hippocampus, formix, cerebellum, brain stem, medulla, pons, basal ganglia, globus pallidum, striatum, spinal cord, ganglia, cranial nerves, peripheral nerves, retina, or cochlea. In another embodiment, a neural system in accordance with the present invention can be any ensemble of one or more neurons, and/or other excitable cells, such as muscle, heart, retinal, cochlear, tissue culture cells, stem or progenitor cells.

In one embodiment, the methods and/or device of this invention perform an electrode-based activity on the neuronal structure and/or system using the spread of the multiple micro-wire electrodes. In another embodiment, methods for measuring and recording neuronal activity can be accomplished according to any suitable method. In another embodiment, the neuronal activity is monitored extra-cellularly by measuring the extra-cellular electrical potential of a target population of neurons. Such measurements can reveal complex spikes or burst activity, sharp or slow waves, epileptiform spikes or seizures, arising from one or more neurons in the neural system. In another embodiment, the neuronal activity can be measured by recording the neural system's electrical potential in the extra-cellular space. In another embodiment, the multi-wire electrodes used to measure the field potential produced by the neural system are referred to as “measuring electrodes” or “recording electrodes.” In another embodiment, the field potentials recorded at a given extra-cellular site will depend on a variety of factors, including the location of the electrode(s) with respect to the soma and dendritic trees, the architecture of the neural system, the perfusion solution, etc.

In some embodiments, in response to the applied electric field, the activity of the neural system can be modified in any desired manner. In one embodiment, the activity can be suppressed, reduced, decreased, diminished, eliminated, counteracted, canceled out, etc., or in another embodiment it can be enhanced, increased, augmented, facilitated, etc. In order to determine whether the activity of the system has been modified, preferably the same neuronal activity measured in the measurement step is re-measured. In another embodiment, the measurement of the neuronal activity is performed simultaneously and continuously with the applied field.

In another embodiment, the recording electrodes can detect the field potential from the applied field as well as the activity generated by the neural system. There are a number of methods that can be used to distinguish the neuronal activity from the applied fields. In one embodiment, the active electrodes are placed in the tissue, preferably near the cell body layer of the target neurons, while the reference electrode is placed preferably in the bath external to the tissue. The values obtained from each electrode can be electronically subtracted from each other, reducing background noise. For in vivo use, the differential measuring electrodes can be placed at the same isopotential with respect to the applied field. The electrodes can be as close to the target population as possible, without damaging it. Other methods to reduce noise and the artifact from the applied field can be used as well, either alone, or in combination with the differential electrodes, including filtering and post-processing of the measured signal.

In one embodiment, the methods of this invention for performing an activity in a neuronal structure comprise inserting the device of this invention into a subject, by optionally a surgical procedure wherein the three dimensional configuration of the micro-wire electrodes is pre-determined in a solution prior to insertion of the device into a subject; releasing and spreading the micro-wire electrodes at a target tissue, wherein the micro-wire electrodes should retain relative positions within the array, wherein these positions are determined with respect to markers and to the distal end of device according to the map of micro-wire electrode tips; and performing an electrode-based activity on the neuronal structure using said spread multiple micro-wire electrodes.

In one embodiment, the determination of the positioning of the tips in a subject is done by triangulation. In another embodiment, the positioning is done by radiovisiography. In another embodiment, the positioning is done by ultrasonography. In another embodiment, the positioning is done by any other method. In another embodiment, the three-dimensional configuration of tips of the micro-wire electrodes can be mapped onto a calibrated three-dimensional image of a brain using modeling and imaging software (for example, 3-D Doctor). In another embodiment, stereotactic locations of the tips may be determined directly by neurosonography, MRI, stereotactic X-ray imaging, or any other known method.

In one embodiment, a current is introduced into each of the micro-wire electrodes for determining the desired configuration of the micro-wire electrodes, prior to insertion of the device into a subject. In another embodiment, the current is between 5-10 μA and it is introduced for 1-3 seconds.

In one embodiment, the precise locations of tips may also be determined by the formation of electrolytic lesions. In another embodiment, electrolytic lesions are obtained when a DC of between 5-10 μA for 8 seconds is passed through the tips and small lesions of approximately 50-100 μm are produced. These lesions can then be visualized by staining. In another embodiment, electrolytic lesions are obtained when a DC of between 5-15 μA is passed through the tips. These lesions can then be visualized by staining. In another embodiment, electrolytic lesions are obtained when a DC of between 5-50 μA is passed through the tips. These lesions can then be visualized by staining. In another embodiment, electrolytic lesions are obtained when a DC of between 10-20 μA is passed through the tips. These lesions can then be visualized by staining. In another embodiment, electrolytic lesions are obtained when a DC of between 25-30 μA is passed through the tips. These lesions can then be visualized by staining. In another embodiment, electrolytic lesions are obtained when a DC of between 30-50 μA is passed through the tips.

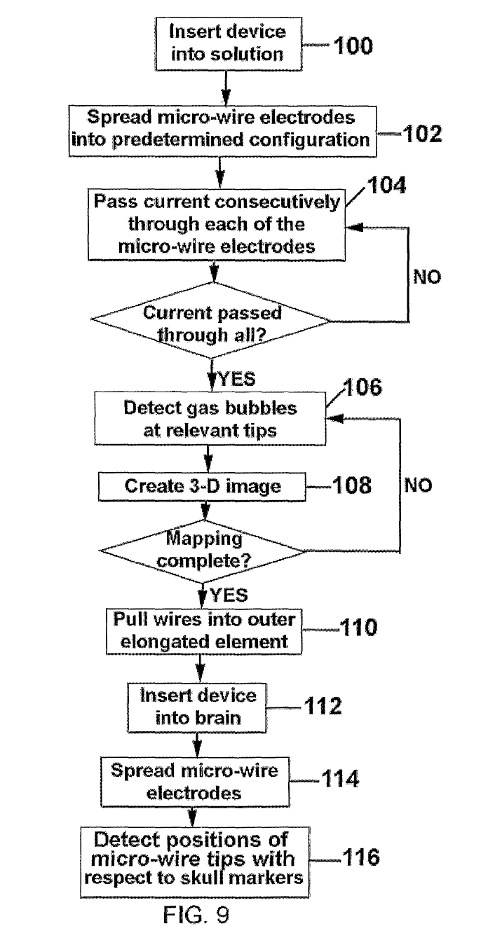

In one embodiment, FIG. 9 depicts a flow-chart diagram illustration of some methods of this invention. In one embodiment, device 10 is inserted (step 100) into a solution, such as NaCl solution (0.95% in water). Micro-wire electrodes are spread apart (step 102) into a desired configuration. Next, a current is introduced (step 104) into each of the micro-wire electrodes 30. This is done consecutively until all of micro-wire electrodes 30 have been stimulated. Gas bubbles form at tip 46. These gas bubbles can be detected (step 106) using, for example, an optoelectronic setup with two digital microscopes (for example, Labomed Digi Star, Mel Sobel Microscopes Ltd. International Falls, Minn.). 2-D images of the electrodes with gas bubbles are created in two orthogonal planes (step 108). When all of the bubbling micro-wire electrode tips 46 have been detected and mapped, micro-wire electrodes 30 are retracted into outer elongated element 22 (step 110) and device 10 is removed from solution. Device 10 may then be inserted (step 112) into a brain for a surgical procedure. It should be readily apparent that when micro-wire electrodes 30 are released and spread (step 114) at the target tissue within the brain, they should retain relative positions within the array of micro-wire electrodes 30. These positions may then be determined (step 116) with respect to skull markers and to the distal end 24 of device 10 according to the map of micro-wire electrode tips 46.

In one embodiment, this invention provides a method of using the device of this invention comprising inserting the device into a subject, advancing the device into close proximity with a targeted neuronal structure, releasing multiple micro-wire electrodes from the outer elongated element and/or body, spreading the multiple micro-wire electrodes into a configuration within the neuronal structure, and performing some type of electrode-based activity on the neuronal structure using the spread multiple micro-wire electrodes. The configuration may be pre-determined prior to insertion of the device into the subject. The releasing may be done by pushing inner elongated element and/or body distally with respect to outer elongated element and/or body, thus causing the multiple micro-wire electrodes to exit at the distal end of outer elongated element and/or body.

In one embodiment, the methods and/or device of this invention are used to record normal and/or abnormal neuronal activity in a subject. In one embodiment, the methods and/or device of this invention are used to determine the locations of abnormal activity and to calculate their stereotactic coordinates. Once the micro-wire electrodes of this invention obtain the relevant information, they are retracted into the outer elongated element and/or body, and the device is removed from the subject. A standard permanent stimulating electrode may then be introduced and placed according to the determined stereotactic coordinates of the site with abnormal activity.

In one embodiment, the methods and/or device of this invention are used to stimulate nervous tissues. In another embodiment, the methods and/or device of this invention are used to stimulate brain tissues. Such stimulations may be useful for diagnostic purposes. In another embodiment, such stimulation may be useful for diagnosing Parkinson's disease, epilepsy and other movement disorders. In another embodiment such stimulation may be useful in diagnosing cognitive functions or dysfunctions, dementia or Alzheimer's disease. In another embodiment, such stimulation comprises applying current/voltage via the multiple micro-wire electrodes in the range of current 10-20 μA, frequency 100-200 Hz, and pulse width 40-60 μsec. Due to the high current densities near the tips, the strength and duration of the pulses of biphasic current used should be such that excessive tissue damage is prevented. In one embodiment, this invention provides a methods and/or device for modulating the neuronal activity of a neural system comprising neurons, such as a brain, brain regions, or any in vivo or in vitro collection of neurons. In another embodiment, this invention involves the use of applied electric fields to modulate the behavior of a target neural system. In another embodiment, the polarity and magnitude of the applied electric field is varied according to information gathered from the modulated neural system, or any other desired source chosen to provide feedback, to modulate the strength of the applied electric field.

In one embodiment, at least one micro-wire electrode is plated by porous gold and is chronically implanted in a subject and can be used to treat a disease. In one embodiment porous gold plated electrode tips of the implanted device can be used to treat diseases of the nervous system, to obtain neuronal structure, to restore neuronal function, paralysis, and motor and sensory deficits to enhance or suppress neuronal activity and associated phenotypes, and the like. In one embodiment, the implanted micro-wire electrode is coated or plated by a biocompatible material other than porous gold.

In some embodiments, the methods and/or device of the present invention are useful for treating brain diseases characterized by aberrant neuronal activity. Epilepsy, for instance, is a brain disorder characterized by recurrent seizures, affecting 1-2% of the population. In epilepsy, the pattern of neuronal discharge becomes transiently abnormal. In one embodiment, an application of the device using adaptive electric field can be used to suppress the epileptiform activity, effectively treating and controlling the brain disorder, wherein the multiple wire electrodes can be placed over the neocortex in the subdural, subarachnoid, or epidural spaces, or within the sulci of the brain. In another embodiment, any brain disorder that displays abnormal activity, such as oscillatory or pulsating activity, can be treated analogously, such diseases, include, schizophrenia, depression (unipolar or bipolar), Parkinson's disease, anxiety or obsessive-compulsive disorder (OCD), where the electric field is applied to the particular brain region exhibiting the abnormal activity, for example to the cortex, hippocampus or thalamus.

In another embodiment, Parkinson's disease is characterized by decreased activity in cells that produce dopamine. Patients with the disease experience tremors, rigidity, and difficulty in movement. Patients with Parkinson's disease can be treated by applying an electric field in an amount effective to ameliorate one or more symptoms of the disease. In one embodiment the field electrodes of the present invention can be placed in any suitable region of the brain, such as the thalamus or basal ganglia. The amount of applied field can be changed in response to an electrical activity in the brain, or in response to a manifestation of such electrical activity. The field can be applied until one or more symptoms are eliminated, such as tremors or difficulty in initiating movement.

In one embodiment, the multi-wire electrodes of the present invention are made of or coated by a biocompatible material. In one embodiment biocompatible coating or material enable the permanent, chronic or long periods implanting of multi-wire electrodes in a subject body part. In one embodiment such implant also relates to restoring or repairing a brain function. These functions include, for example, not limited to, sensory functions, such as vision, hearing, smell, touch, and taste, motor activity and function, or somatic activity and function. In another embodiment, the methods and/or device can be useful to treat a condition where an animal or human has lost its vision due to a peripheral defect, such as the loss of an eye, but the visual cortex is largely intact. In another embodiment, the methods and/or device of this invention can be used to restore vision by creating patterned activity in the brain using an applied field. In another embodiment, the methods and/or device of this invention can be used to restore other lost functions, e.g., hearing or touch to the auditory or somatosensory cortex, respectively.

In some embodiments, neuronal structure experimental studies have shown that pathological processes can change the integrative behavior of the cardiac neuraxis. In one embodiment, the device of this invention is biocompatible and can be implanted in a subject. In one embodiment the present invention provides a methods and/or a biocompatible and implantable device for identifying cardiac neuraxis. In another embodiment, the present invention utilizes a device of this invention for spinal cord stimulation to alter and/or affect the peripheral cardiac nervous system and thereby protect cardiac function.

In some embodiments, this invention makes use of the methods and/or device for detecting changes in the levels and distribution of neuronal activity caused by proposed therapeutic agents. This information has important predictive value in determining the potential effects of a proposed therapeutic agent in humans. Comparisons of subject animals and/or humans treated with the agent of interest with subject animals and/or a human treated with known agents, or not treated at all, allows the identification of new therapeutic agents. Such a method is especially useful for identifying agents that can be used therapeutically and/or prophylactically in brain disease. In another embodiment, the following agents can be administered to the neural system, comprising, e.g., neurotransmitter agonists and antagonists (such as, serotonin, dopamine, gaba, glutamate), sympathomimetics, cholinergics, adrenergics, muscarinics, antispasmodics, hormones, peptides, genes (sense and anti-sense, including genetic therapy), metabolites, cells (e.g., where neural grafting is being used as a modulatory therapy), sedatives, hypnotics, anti-epileptics (e.g., acetazolamide, amphetamine, carbamazepine, chloropromazine, clorazepate, dextroamphetamine, dimenhydrinate, ephedrine, divalproex, ethosuximide, magnesium sulfate, mephenytoin, metharbital, methsuximide, oxazepam, paraldehyde, pamethadione, phenacemide, phenobarbital, phensuximide, phenytoin, primidone, trimethadione, valproate, etc.), hormones or peptides. In some embodiments, such agent administration is conducted in conjunction with, is following, or followed by the use of methods and device of this invention.

The following examples are presented in order to more fully illustrate the preferred embodiments of the invention. They should in no way be construed, however, as limiting the broad scope of the invention.

### EXAMPLES

### Example 1

### Operation and Uses of Multi-wire Micro Electrodes

A device such as the ones described in the various embodiments above was provided. The device included 8 micro-wire electrodes having wire diameters of 25-33 μm, an outer tube fabricated from a standard hypodermic or thin wall stainless steel tube having a diameter of ˜0.6 mm, and an inner tube (also fabricated from a standard hypodermic or thin wall stainless steel tube) having a diameter of ˜0.4 mm, so that the bent micro-wires would have room to move freely inside the outer guide tube. The proximal ends of the micro-wires were soldered to the contacts of a miniature connector. In chronic experiments with behaving rats, the devices were attached to a miniature micro-drive, the combined weight of which did not exceed 2 g.

Prior to in vivo experiments, the ability of the micro-wire electrodes to spread was examined in a gel that imitates the mechanical properties of brain tissue.

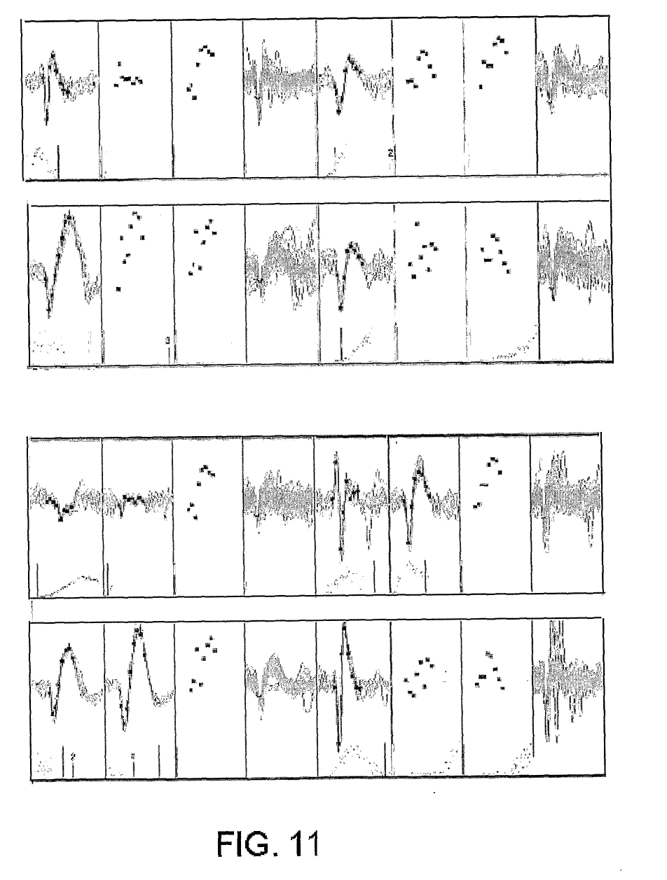

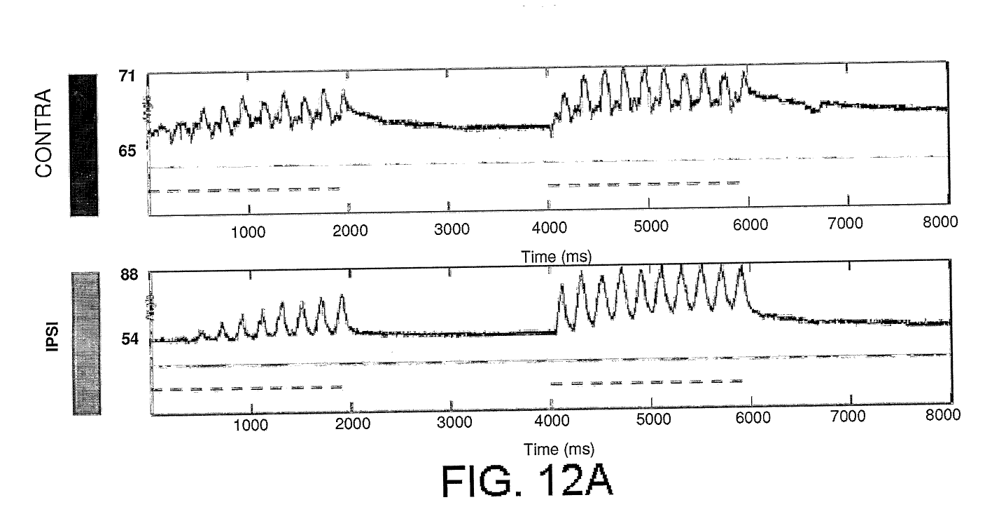

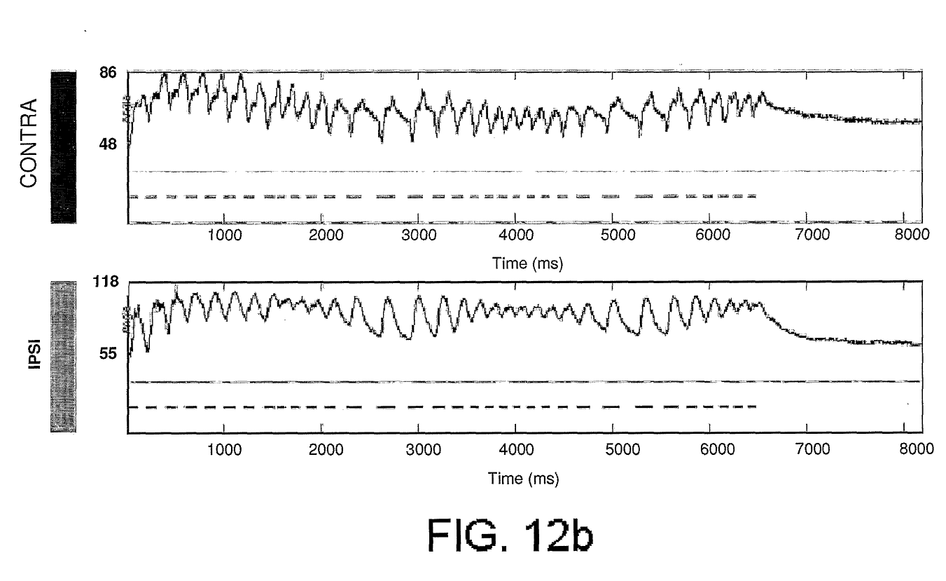

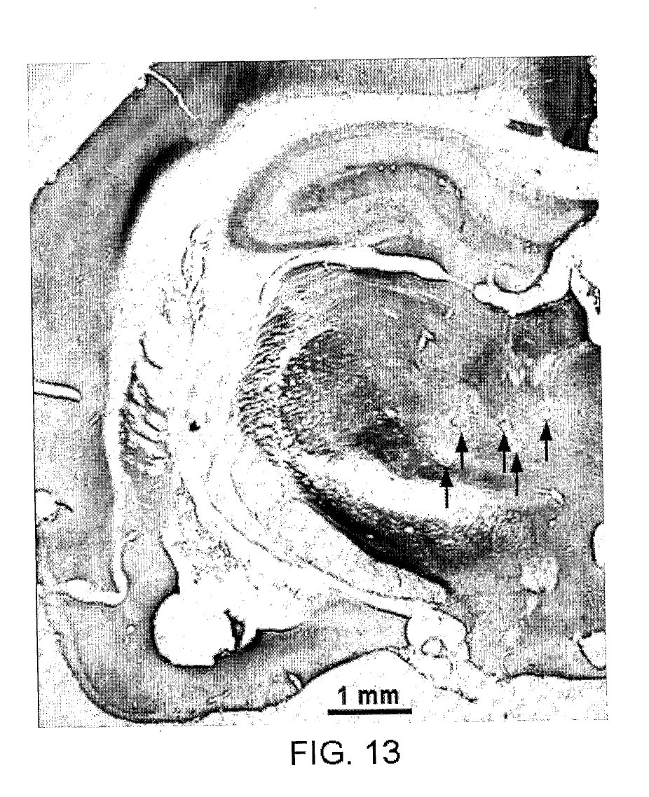

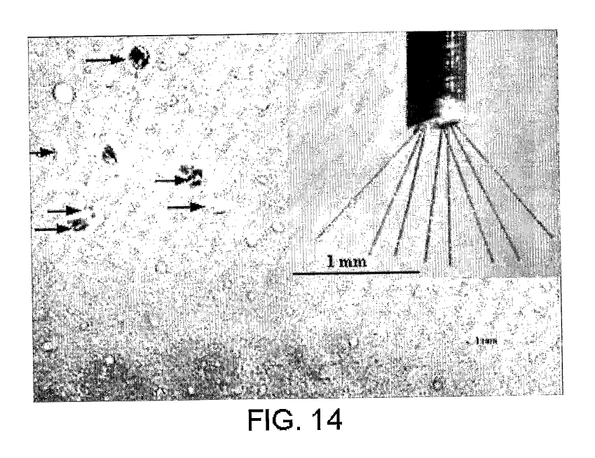

Next, the ability of micro-wire electrodes to spread was tested in live brain tissue in rats. Rat thalamic nuclei were chosen as a target, and the device was inserted stereotaxically through a trephine hole (1 mm in diameter) in the skull, drilled 2 mm lateral to midline and −3.5 mm caudal to the bregma. The device was attached to a stereotaxic micromanipulator and the guide was inserted into the brain vertically to a depth of ˜4 mm from the dura, after which the micro-wires were spread within the thalamic nuclei. Extra-neuronal recordings, electrical stimulation (100-400 Hz, 40 μs, 10-20 μA), and electrolytic (10 μA, 2 pulses of 4 s) lesions were produced. Examples of single- and multi-unit recordings from POm thalamic nucleus of a rat are demonstrated in FIG. 11. Examples of behavioral responses to deep brain stimulation of relevant thalamic nuclei in the rat vibrissal pathway are shown in FIGS. 12A and 12B [red dashed lines are stimulus applications, black curves are whisker deflections that were registered with a fast digital video camera (MotionScope PCI 1000; Redlake, San Diego, Calif., USA) contralateral (CONTRA) and ipsilateral (IPSI)]. Brain slices 60 μm thick were later cut, stained and examined histologically, as shown in FIG. 13, which is a histology sample showing these lesions in a rat brain. Five lesions are seen in a single 60 μm thick coronal section stained for cytochrome oxidase activity (arrows). In this case, the micro-wire electrode configuration was similar to a biconvex lens, which was oriented coronally. This configuration allowed for coverage of the volume of elongated brain nuclei (ventral posteromedial thalamic nucleus or posterior thalamic nuclear group).

In a different configuration specifically constructed for somatosensory recordings, the distribution of the electrodes was radial relative to the guide axis. As described above, a trephine hole was made in the skull, the guide was inserted so as to touch the dura, the dura under the guide was carefully cut, and the electrodes introduced into layer IV (˜0.7 mm from the surface of the brain) of the rat somatosensory cortex. After MER of neuronal activity, current (10 μA; 4×2 s) was delivered through the tips of each electrode to produce electrolytic lesions, as shown in FIG. 14.

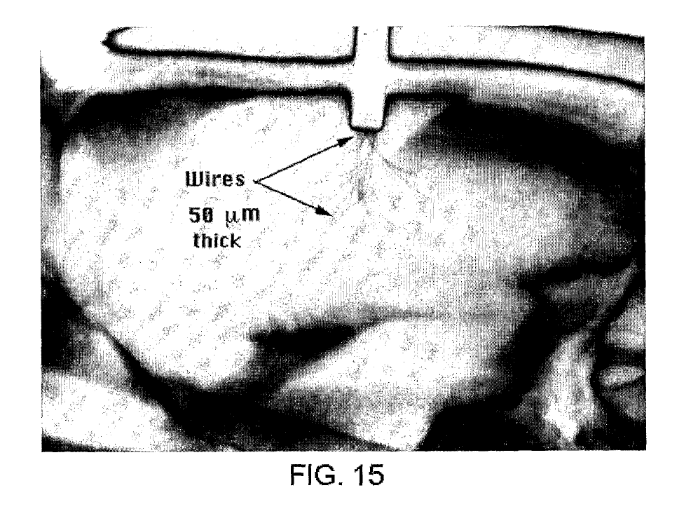

The spread of the micro-wire electrodes of a device introduced into a rat skull through a trephine hole was visualized directly by radiovisiography. Radiovisiography was performed with a RVG Ultimate Imaging System (Trophy Imaging Co.; Marne la Vallee CEDEX2, France) that combines high resolution imaging with maximum reduction in X-ray doses. In the resulting radiovisiogram, depicted in FIG. 15, eight wire electrodes (50 μm in diameter) are seen emerging from the tip of the outer elongated element 22, and the positions of the distal ends of each can be precisely determined.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While certain features of the present invention have been illustrated and described herein, many modifications, substitutions, changes, and equivalents may occur to those of ordinary skill in the art. It is, therefore, to be understood that the appended claims are intended to cover all such modifications and changes as fall within the true spirit of the present invention.

1. A device for recording and measuring activity of nervous tissue, the device comprising: an inner elongated element comprising an inner elongated element distal end, an inner elongated element proximal end, and an inner elongated element body extending from said inner elongated element distal end to said inner elongated element proximal end, said inner elongated element body defining a longitudinal axis; an outer elongated element comprising an outer elongated element distal end, an outer elongated element proximal end, and an outer elongated element body extending from said outer elongated element distal end to said outer elongated element proximal end, said outer elongated element positioned concentrically with respect to said inner elongated element, wherein said inner elongated element is movable with respect to said outer elongated element along said longitudinal axis; and multiple micro-wire electrodes, each of said multiple micro-wire electrodes comprising an attached portion, said attached portion at least partially attached to said inner elongated element, and a free portion, said free portion positionable within said outer elongated element body having tips at distal ends of said electrodes, and electrical activity of neurons from said electrode tip is recorded.

2. The device of claim 1, wherein said inner elongated element is a tubular element and said inner elongated element body has a tubular shape.

3. The device of claim 1, wherein said outer elongated element is a tubular element and said outer elongated element body has a tubular shape.

4. The device of claim 1, wherein said multiple micro-wire electrodes comprise 4-30 micro-wire electrodes.

5. The device of claim 4, wherein said multiple micro-wire electrodes comprise 8-16 micro-wire electrodes.

6. The device of claim 1, wherein said free portion is 1-6 mm in length.

7. The device of claim 6, wherein said free portion is 3-4 mm in length.

8. The device of claim 1, wherein said free portion of at least some of said multiple micro-wire electrodes differs in length from said free portion of others of said multiple micro-wire electrodes.

9. The device of claim 1, wherein upon movement of inner elongated element with respect to said outer elongated element, said free portion is releasable from said outer elongated element.

10. The device of claim 1, wherein said multiple micro-wire electrodes are comprised of an Iridium-Platinum alloy.

11. The device of claim 1, wherein said multiple micro-wire electrodes are individually insulated.

12. The device of claim 11, wherein said multiple micro-wire electrodes comprise polyimide insulation.

13. The device of claim 1, wherein said multiple micro-wire electrodes are comprised of a shape memory alloy and wherein said free portion is pre-shaped.

14. The device of claim 1, wherein said multiple micro-wire electrodes are comprised of a material having high plasticity such that pre-deformation of said material results in permanent deformation.

15. The device of claim 1, wherein said multiple micro-wire electrodes are less than 40 μm in diameter.

16. The device of claim 15, wherein said multiple micro-wire electrodes are 25-33 μm in diameter.

17. A method for recording an activity in a neuronal structure, the method comprising the following steps: inserting a device comprising multiple micro-wire electrodes enclosed in an enclosure into a subject; advancing said device into close proximity with the neuronal structure; releasing said multiple micro-wire electrodes having tips at distal ends thereof from said enclosure; spreading said multiple micro-wire electrodes into a configuration at the neuronal structure; recording single- and multi-unit neuronal activity; and performing an electrode-based activity on the neuronal structure using said spread multiple micro-wire electrodes, and electrical activity of neurons from said electrode tip is recorded.

18. The method of claim 17, wherein said enclosure is an outer tube and wherein said device further comprises an inner tube positioned within and movable with respect to said outer tube, said inner tube connected to said multiple micro-wire electrodes, and wherein said releasing comprises pushing said inner tube so as to release said multiple micro-wire electrodes.

19. The method of claim 17, wherein said spreading comprises pre-shaping said multiple micro-wire electrodes into a pre-determined configuration prior to enclosing said multiple micro-wire electrodes in said enclosure, such that upon said releasing of said multiple micro-wire electrodes from said enclosure, said pre-shaped multiple micro-wire electrodes maintain said pre-determined configuration.

20. (canceled)

21. The method of claim 17, wherein said performing comprises stimulating said neuronal structure.

22. The method of claim 17, wherein said performing comprises creating lesions in said neuronal structure.

23. The method of claim 17, wherein said performing comprises spatial mapping of patterns of neuronal activity.

24. The method of claim 23, wherein said spatial mapping comprises spatial mapping of abnormal activity.

25. The method of claim 23, wherein said spatial mapping comprises: prior to said inserting said device into a subject, releasing said multiple micro-wire electrodes from said enclosure; spreading said multiple micro-wire electrodes into a configuration; and determining a three-dimensional map of tips of said multiple micro-wire electrodes; and wherein after said inserting said device into the body, said spreading results in a similar configuration as said spreading prior to said inserting, thereby enabling spatial mapping of neuronal activity onto said determined three-dimensional map.

26. The method of claim 17, wherein said neuronal structure is a subthalamic nucleus of a brain.

27. The method of claim 17, wherein said performing comprises retraction of said released micro-wire electrodes having tips at distal ends thereof, rotation of said inner elongated element by few degrees, and repeated release of said three electrodes in a new direction; repeated electrode retraction, rotation, and release in new directions allows to scan the whole volume of a desired brain structure without device extraction and additional guide penetrations.
